(19) United States
(12) Patent Application Publication
(10) Pub. No.: US 2003/0235606 A1
(43) Pub. Date: Dec. 25, 2003

(54) Oral hygiene products and methods of making oral hygiene products

(76) Inventor: Kenneth H. Nussen, Santa Monica, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025, US

(21) Appl. No.: 10/373,250

(22) Filed: Feb. 24, 2003

Related U.S. Application Data
(63) Continuation-in-part of application No. 10/177,535, filed on Jun. 21, 2002.

(51) Int. Cl. A61K 9/70; A61K 47/36; A61K 35/78

(52) U.S. Cl. 424/443; 514/777; 424/725

(57) ABSTRACT

An edible film formed from natural ingredients and containing green tea extract and adapted to dissolve in the mouth of a consumer. A method including mixing one or more natural film forming agent(s) with green tea extract (GTE) to form a composition and drying the composition on a substrate to form an edible film.

### CROSS-REFERENCE TO RELATED APPLICATION

The application is a continuation-in-part of co-pending application Serial No. 10/177,535, filed Jun. 21, 2002, by Kenneth H. Nussen, entitled DENTAL HYGIENE PRODUCTS AND METHODS OF MAKING DENTAL HYGIENE PRODUCTS.

### BACKGROUND

1. Field

Oral hygiene products including dental floss and edible dental film composed of natural ingredients.

2. Background

Oral hygiene products include toothbrushes, dental floss, mouthwash and edible film. These products deliver antibacterial and odor masking substances to reduce bad breath, and tartar control substances that protect teeth and counteract plaque build up. These products typically use artificial active ingredients such as fluoride, triclosan, and xylitol and other artificial substances such as artificial sweeteners, flavorants and coloring.

Edible films deliver an active breath freshening agent along with flavorants and color additives. The structure of an edible film is typically composed of pullulon or hydropropylmethyl cellulose. The active breath freshening agent is typically methyl salicate, thymol, eucalyptol or menthol. Color additives include artificial food colorings (e.g., FD&C green No. 3). Thus, typical edible films are primarily composed of artificial substances, and rely on artificial substances for their structure and breath freshening properties.

An oral hygiene product incorporating tea tree oil can provide an antibacterial effect that may help to reduce bad breath and retard the development of plaque. Tea tree oil has several drawbacks, however, including its strong medicinal odor and its inadequacy at preventing inflammation of the gums. These factors have contributed to the unavailability of a tea tree oil oral hygiene product that is both pleasant to use and has anti-inflammatory properties.

Green tea extract is derived from the leaves of the plant Camellia sinensis and is rich in antioxidants. Green tea extract is commonly available in pill form as a nutritional supplement. Green tea extract has been shown to be effective at fighting cavities, prohibiting the growth of oral bacteria, and reducing inflammation of the gums.

### BRIEF DESCRIPTION OF THE DRAWINGS

The claims are illustrated by way of example and not by way of limitation in the figures of the accompanying drawings in which like references indicate similar elements. It should be noted that references to “an” or “one” embodiment in this disclosure are not necessarily to the same embodiment, and such references mean at least one.

FIG. 1 is a view of a single use package of edible film.

FIG. 2 is a view of a multiple use package of edible film.

### DETAILED DESCRIPTION

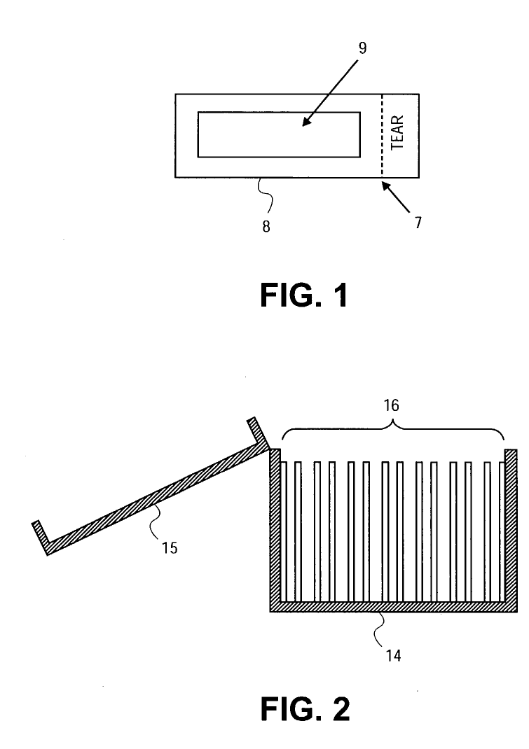

Edible film is made by combining one or more film forming agents with one or more active ingredients. The film composition may be heated to promote dissolution of the ingredients and subsequent drying. The film forming agents and the active ingredients must be in amounts that are non-toxic and safe for human consumption. In one embodiment, the active ingredients may be combined and optionally hydrated before being added to the film composition. The resulting film composition is then transferred to a suitable substrate where it dries to form a thin film. Drying can be accelerated by exposing the film composition to heated air or by heating the substrate. When dried, the film is cut into strips of suitable size and packaged. Representative packaging includes, but is not limited to, a spool, a single use package (FIG. 1), and a multiple use package (FIG. 2).

The film forming agents enable the film composition to dry into a pliable film without interfering with or diminishing the effectiveness of the active ingredients. In one embodiment, one or more film-forming agents such as pullulan, xanthan gum, locust bean gum, and carrageenan are combined with an active ingredient, such as an amount of green tea extract (GTE) that is effective at reducing oral bacteria and fighting cavities. A suitable amount includes, but is not limited to, about two to five percent by weight GTE in a batch. In another embodiment, GTE is combined with tea-tree oil (TTO) in an amount sufficient to mask the odor and taste of the TTO.

Flavoring (e.g., mint, cinnamon, bubble gum, anise, etc.), coloring, and sweeteners may optionally be added to the film composition. In one embodiment, one or more sweeteners such as saccharin, fructose, sucrose, maltose, and corn syrup are added to the film composition. The flavoring, coloring and sweeteners may be combined before being added to the film composition. In another embodiment, mint flavor is added to the film composition, for example, in an amount of approximately 10 percent by weight of the composition.

The film composition may be formed by hydrating (if necessary) one or more film forming agents to create a film composition. To the film composition is added freeze-dried GTE commercially available from XEL Herbaceuticals of Salt Lake City, Utah. In an embodiment where the edible film also includes TTO, TTO liquid commercially available from Natural extracts of Australia of Los Angeles, Calif. is added to the film composition.

In one embodiment, the film composition is entirely formed of natural ingredients. Artificial sweetners, colorings and similar substances are not used. In one embodiment, the composition includes sodium alginate derived from brown seaweed that functions as a thickener. Anise oil or peppermint oil, mint or similar naturally occurring spices and substances or combinations thereof are used as flavorants. Anise oil also has antibacterial properties. Chlorophyll or similar substances may be used in the composition as a natural coloring. Chlorophyll and other natural coloring substances are derived from vegetables, plants, fruits and similar natural sources. Tapioca starch may be used as natural water binding agent and texturizing agent. In one embodiment, stevia extract or similar substance may be used as a sweetener. Carrageenan derived from seaweed serves as a natural film forming agent. GTE is used in the all natural composition and provides anti-cavity, antibacterial, and anti-inflammatory properties. In one embodiment, additional ingredients may include spinach extract, purified water, sorbitan acid ester and ascorbic acid. Each of these substances is derived from a naturally occurring source.

The following table is an exemplary composition of the natural ingredient composition:

| Item | Dry Weight |
|---|---|
| GTE | 0.1-8% |
| Flavoring Oil | 1-15% |
| (Peppermint/Anise) | |
| Sodium Alginate | 35-55% |
| Natural Color | 0.1-3% |
| (Chlorophyll) | |
| Tapioca Starch | 8-45% |
| Stevia Extract | 1-7% |
| Carrageenan | 4-6% |
| Glycerin | 15-35% |
| Sorbitan Acid Ester | 1-3% |

After combining the ingredients, the film composition is then transferred onto a suitable substrate. A suitable substrate is nonporous and will not react chemically with the film composition. As the film composition dries on the substrate, it changes state from a liquid to a pliable film.

The film is then cut into strips and packaged. The film is used by consumers in the following manner. A consumer will remove a piece of film from its packaging and place the film on their tongue. The film will dissolve as it comes in contact with saliva, thereby releasing active ingredients such as GTE and TTO.

FIG. 1 is a view of a single use package of edible film. The edible film 9 is contained within package 8. Package 8 can be opened by tearing along perforation 7. The film 9 is then removed from the package 8 and placed in the mouth of a consumer (e.g.,) on the tongue were it will dissolve.

FIG. 2 is a view of a multiple use package of edible film. The package is comprised of body 14 and lid 15. Lid 15 may be detached from body 14. Inside body 14 are an array of edible strips 16, such that a consumer can carry a quantity of strips in a single container.

It is appreciated that in alternative embodiments various other edible strips can be formed to provide a desirable substance or substances to a user. For example, edible strips can be formed that include one or more vitamins that are necessary for or improve nutrition, or that act as coenzymes and precursors of coenzymes in the regulation of metabolic processes. Representative vitamins include, but are not limited to, Vitamin A, beta kerotene, Vitamin B, Vitamin B6, Vitamin B12, folic acid or folate, Vitamin C, Vitamin D, Vitamin E (including tocopherols) and or tocotrienols. Edible strips may additionally or alternatively include minerals such as calcium, zinc, or similar materials. For example, an edible strip may include an amount of a zinc compound such as zinc gluconate or other zinc compound in an amount sufficient to inhibit symptoms associated with a common cold. In one embodiment, the edible strip includes up to 100 percent of the recommended daily allowance of one or more vitamins or minerals. In this manner, an edible strip provider, an alternative intake form for administering vitamins and minerals (e.g., as opposed to tablets or capsules). Still further, edible strips may additionally or alternatively include various other herbs (e.g., herb extracts) such as ginko biloba, echinacea, kava kava or similar extracts.

The specification has been described with reference to specific embodiments thereof. It will, however be evident that various modifications and changes can be made thereto without departing from the broader spirit and scope of the appended claims. The specification and drawings are, accordingly, to be regarded in an illustrative rather than a restrictive sense.

What is claimed is:

1. A method comprising:
mixing one or more natural film forming agent(s) with at least one of a vitamin, a mineral and an herb to form a composition;
drying the composition on a substrate to form an edible film.

2. The method of claim 1, wherein the one or more film forming agent(s) are selected from the group consisting of sodium alginate and carrageenan.

3. The method of claim 1, further comprising:
cutting the edible film into strips suitable for packaging.

4. The method of claim 1, further comprising:
packaging the edible film strips, wherein the package is one of a spool, a single use package, and a dispenser for pre-cut strips.

5. The method of claim 1, further comprising:
mixing one or more natural sweeteners with the natural film forming agents(s).

6. The method of claim 5, wherein the natural sweetener is stevia extract.

7. The method of claim 1, further comprising:
mixing a natural coloring with the natural film forming agent(s).

8. The method of claim 7, wherein the natural coloring agent is chlorophyll.

9. The method of claim 1, wherein the herb is a green tea extract.

10. An edible film produced according to the method of claim 1.

11. A method of delivering an anti-cavity and anti-inflammatory ingredients by introducing the edible film of claim 1 into an oral cavity.
